SEQUENCE LISTING (41 sequences)

SEQ ID NO: 1 - protein, 4 aa, Artificial Sequence
  Description of Artificial Sequence Synthetic peptide
KKKK

SEQ ID NO: 2 - protein, 8 aa, Artificial Sequence
  Description of Artificial Sequence Synthetic peptide
KKKKKKKK

SEQ ID NO: 3 - protein, 12 aa, Artificial Sequence
  Description of Artificial Sequence Synthetic peptide
KKKKKKKKKKKK

SEQ ID NO: 4 - protein, 16 aa, Artificial Sequence
  Description of Artificial Sequence Synthetic peptide
KKKKKKKKKKKKKKKK

SEQ ID NO: 5 - protein, 4 aa, Artificial Sequence
  Description of Artificial Sequence Synthetic peptide
RRRR

SEQ ID NO: 6 - protein, 8 aa, Artificial Sequence
  Description of Artificial Sequence Synthetic peptide
RRRRRRRR

SEQ ID NO: 7 - protein, 12 aa, Artificial Sequence
  Description of Artificial Sequence Synthetic peptide
RRRRRRRRRRRR

SEQ ID NO: 8 - protein, 16 aa, Artificial Sequence
  Description of Artificial Sequence Synthetic peptide
RRRRRRRRRRRRRRRR

SEQ ID NO: 9 - protein, 4 aa, Artificial Sequence
  Description of Artificial Sequence Synthetic peptide
KRKR

SEQ ID NO: 10 - protein, 4 aa, Artificial Sequence
  Description of Artificial Sequence Synthetic peptide
KKKR

SEQ ID NO: 11 - protein, 12 aa, Artificial Sequence
  Description of Artificial Sequence Synthetic peptide
KKKRRRKKKRRR

SEQ ID NO: 12 - protein, 16 aa, Artificial Sequence
  Description of Artificial Sequence Synthetic peptide
KKKKRRRRKKKKRRRR

SEQ ID NO: 13 - protein, 7 aa, Artificial Sequence
  Description of Artificial Sequence Synthetic peptide
XRXLRRX

SEQ ID NO: 14 - protein, 7 aa, Artificial Sequence
  Description of Artificial Sequence Synthetic peptide
PKKKRKV

SEQ ID NO: 15 - protein, 25 aa, Artificial Sequence
  Description of Artificial Sequence Synthetic peptide
LVRKKRKTEEESPLKDKDAKKSKQE

SEQ ID NO: 16 - protein, 12 aa, Artificial Sequence
  Description of Artificial Sequence Synthetic peptide
PEVKKKRKPEYP

SEQ ID NO: 17 - protein, 16 aa, Artificial Sequence
  Description of Artificial Sequence Synthetic peptide
XXVXLLXXVLLXLLXX

SEQ ID NO: 18 - protein, 16 aa, Artificial Sequence
  Description of Artificial Sequence Synthetic peptide
AAVALLPAVLLALLAP

SEQ ID NO: 19 - protein, 45 aa, Artificial Sequence
  Description of Artificial Sequence Synthetic polypeptide
AAVALLPAVLLALLAPMSVLTPLLLRGLTGSARRLPVPRAKIHSL

SEQ ID NO: 20 - protein, 39 aa, Artificial Sequence
  Description of Artificial Sequence Synthetic polypeptide
KKKKKKKKKKKKKKKKPKKKRKVAAVALLPAVLLALLAP

SEQ ID NO: 21 - protein, 23 aa, Artificial Sequence
  Description of Artificial Sequence Synthetic peptide
AAVALLPAVLLALLAPPKKKRKV

SEQ ID NO: 22 - protein, 116 aa, Artificial Sequence
  Description of Artificial Sequence Synthetic polypeptide
KKKKKKKKKKKKKKKKKKKKXXVXLLXXVLLXLLXXXXVXLLXXVLLXLLXXXXVXLLXXVLLXLLXXXXVXLLXXVLLXLLXXXXVXLLXXVLLXLLXXXXVXLLXXVLLXLLXX

SEQ ID NO: 23 - protein, 212 aa, Artificial Sequence
  Description of Artificial Sequence Synthetic polypeptide
XXVXLLXXVLLXLLXXXXVXLLXXVLLXLLXXXXVXLLXXVLLXLLXXXXVXLLXXVLLXLLXXXXVXLLXXVLLXLLXXXXVXLLXXVLLXLLXXKKKKKKKKKKKKKKKKKKKKXXVXLLXXVLLXLLXXXXVXLLXXVLLXLLXXXXVXLLXXVLLXLLXXXXVXLLXXVLLXLLXXXXVXLLXXVLLXLLXXXXVXLLXXVLLXLLXX

SEQ ID NO: 24 - protein, 49 aa, Artificial Sequence
  Description of Artificial Sequence Synthetic polypeptide
KKKKAAVALLPAVLLALLAPMSVLTPLLLRGLTGSARRLPVPRAKIHSL

SEQ ID NO: 25 - protein, 53 aa, Artificial Sequence
  Description of Artificial Sequence Synthetic polypeptide
KKKKKKKKAAVALLPAVLLALLAPMSVLTPLLLRGLTGSARRLPVPRAKIHSL

SEQ ID NO: 26 - protein, 57 aa, Artificial Sequence
  Description of Artificial Sequence Synthetic polypeptide
KKKKKKKKKKKKAAVALLPAVLLALLAPMSVLTPLLLRGLTGSARRLPVPRAKIHSL

SEQ ID NO: 27 - protein, 61 aa, Artificial Sequence
  Description of Artificial Sequence Synthetic polypeptide
KKKKKKKKKKKKKKKKAAVALLPAVLLALLAPMSVLTPLLLRGLTGSARRLPVPRAKIHSL

SEQ ID NO: 28 - protein, 20 aa, Artificial Sequence
  Description of Artificial Sequence Synthetic peptide
KKKKAAVALLPAVLLALLAP

SEQ ID NO: 29 - protein, 24 aa, Artificial Sequence
  Description of Artificial Sequence Synthetic peptide
KKKKKKKKAAVALLPAVLLALLAP

SEQ ID NO: 30 - protein, 28 aa, Artificial Sequence
  Description of Artificial Sequence Synthetic peptide
KKKKKKKKKKKKAAVALLPAVLLALLAP

SEQ ID NO: 31 - protein, 32 aa, Artificial Sequence
  Description of Artificial Sequence Synthetic polypeptide
KKKKKKKKKKKKKKKKAAVALLPAVLLALLAP

SEQ ID NO: 32 - protein, 33 aa, Artificial Sequence
  Description of Artificial Sequence Synthetic polypeptide
YKKKKKKKKKKKKKKKKAAVALLPAVLLALLAP

SEQ ID NO: 33 - protein, 48 aa, Artificial Sequence
  Description of Artificial Sequence Synthetic polypeptide
KKKKKKKKKKKKKKKKAAVALLPAVLLALLAPAAVALLPAVLLALLAP

SEQ ID NO: 34 - protein, 44 aa, Artificial Sequence
  Description of Artificial Sequence Synthetic polypeptide
AAVALLPAVLLALLAPKKKKKKKKKKKKAAVALLPAVLLALLAP

SEQ ID NO: 35 - protein, 32 aa, Artificial Sequence
  Description of Artificial Sequence Synthetic polypeptide
KKKKKKKKKKKKKKKKAAVWLLWYVLLFLLYL

SEQ ID NO: 36 - protein, 32 aa, Artificial Sequence
  Description of Artificial Sequence Synthetic polypeptide
KKKKKKKKKKKKKKKKFWVWLLWYVLLFLLYL

SEQ ID NO: 37 - protein, 116 aa, Artificial Sequence; identical to SEQ ID NO: 22
  Description of Artificial Sequence Synthetic polypeptide

SEQ ID NO: 38 - protein, 212 aa, Artificial Sequence; identical to SEQ ID NO: 23
  Description of Artificial Sequence Synthetic polypeptide

SEQ ID NO: 39 - protein, 116 aa, Artificial Sequence; identical to SEQ ID NO: 22
  Description of Artificial Sequence Synthetic polypeptide

SEQ ID NO: 40 - protein, 212 aa, Artificial Sequence; identical to SEQ ID NO: 23
  Description of Artificial Sequence Synthetic polypeptide

SEQ ID NO: 41 - protein, 116 aa, Artificial Sequence; identical to SEQ ID NO: 22
  Description of Artificial Sequence Synthetic polypeptide

(19) United States
(12) Patent Application Publication
(10) Pub. No.: US 2010/0119528 A1
(43) Pub. Date: May 13, 2010

(54) Transport of Biologically Active Molecules into a Cell, Mitochondrion, or Nucleus

(76) Inventors: Gobinda Sarkar, Rochester, MN (US); Mark E. Bolander, Rochester, MN (US); Deendayal Mandal, Kingston, RI (US); Eric W. Mahlum, Rochester, MN (US); Michael J. Yaszemski, Rochester, MN (US)

Correspondence Address: FISH & RICHARDSON P.C., PO BOX 1022, MINNEAPOLIS, MN, 55440-1022, US

(21) Appl. No.: 12/354,142

(22) Filed: Jan. 15, 2009

Related U.S. Application Data
(60) Provisional application No. 61/021,815, filed on Jan. 17, 2008.

(51) Int. Cl. A61K 39/395; C07K 14/00; C07K 7/08; C07K 9/00; C07K 16/00; A61K 38/16; A61K 38/10; A61K 38/14; A61K 39/385; C12N 5/00; C12N 15/00; A61P 43/00

(52) U.S. Cl. 424/178.1; 530/324; 530/326; 530/325; 530/322; 530/391.1; 514/12; 514/13; 514/8; 424/193.1; 435/375; 435/440

(57) ABSTRACT

Novel carrier peptides are described, as well as methods of using the carrier peptides to transport biologically active molecules into a cell, mitochondrion, or nucleus, e.g., by formation of a complex of the carrier peptide non-covalently bound to a biologically active molecule.

### CROSS-REFERENCE TO RELATED APPLICATIONS

This application is claims priority to U.S. Provisional Ser. No. 61/021,815, filed on Jan. 17, 2008, which is incorporated by reference in its entirety herein.

### STATEMENT AS TO FEDERALLY SPONSORED RESEARCH

This invention was made with government support under AR047974 awarded by National Institutes of Health. The government has certain rights in the invention.

### TECHNICAL FIELD

This disclosure relates to carrier peptides, including compositions and methods of using the same.

### BACKGROUND

Poor permeability of most proteins, antibodies, and small molecules across cell membranes compromises their potential as therapeutic reagents. As one example, methods of delivery into the mitochondria are limited. Mitochondria are the “powerhouse” of the cell and are the only source of extra-nuclear DNA in humans. The relatively small human mitochondrial genome (˜16.5 kb) encodes two rRNAs, 22 tRNAs and 13 peptides of the respiratory chain. All other proteins in the mitochondria are encoded by nuclear DNA and transported into the mitochondria. Numerous diseases including cancer are thought to be associated with mitochondrial dysfunction. Thus, an ability to modulate mitochondrial function has important clinical implications.

### SUMMARY

The peptides described herein can function as carrier peptides. These peptides can associate with (e.g., non-covalently bind) biologically active molecules to transport the biologically active molecules into cells and into cell organelles such as nuclei and mitochondria. In some cases, such transport may increase the therapeutic effectiveness of the biological molecules.

Provided herein is a carrier peptide comprising the sequence:

Kₙ-[X₁-X₂-V-X₃-L-L-X₄-X₅-V-L-L-X₆-L-L-X₇-X₈]ₘ

wherein X₁-X₈ are independently L, A, W, F, Y, or V; n is an integer from 4 to 20; and m is an integer from 1 to 6. In some embodiments, the carrier peptide comprises the sequence:

[X₁-X₂-V-X₃-L-L-X₄-X₅-V-L-L-X₆-L-L-X₇-X₈]ₚ-Kₙ-[X₁-
X₂-V-X₃-L-L-X₄-X₅-V-L-L-X₆-L-L-X₇-X₈]ₘ

wherein X₁-X₈ are independently L, A, W, F, Y, or V; n is an integer from 0 to 20; m is an integer from 1 to 6; and p is an integer from 1 to 6. In the carrier peptides described above, in some embodiments, X₁ is A or F; X₂ is A or W; X₃ is A or W; X₄ is P or W; X₅ is A or Y; X₆ is A or F; X₇ is A or Y; and X₈ is P or L.

In certain embodiments the carrier peptide is selected from the group consisting of:

(SEQ ID NO: 24)

K-K-K-K-A-A-V-A-L-L-P-A-V-L-L-A-L-L-A-P-M-S-V-L-T-
P-L-L-L-R-G-L-T-G-S-A-R-R-L-P-V-P-R-A-K-I-H-S-L;

(SEQ ID NO: 25)

K-K-K-K-K-K-K-K-A-A-V-A-L-L-P-A-V-L-L-A-L-L-A-P-M-
S-V-L-T-P-L-L-L-R-G-L-T-G-S-A-R-R-L-P-V-P-R-A-K-I-
H-S-L;

(SEQ ID NO: 26)

K-K-K-K-K-K-K-K-K-K-K-K-A-A-V-A-L-L-P-A-V-L-L-A-L-
L-A-P-M-S-V-L-T-P-L-L-L-R-G-L-T-G-S-A-R-R-L-P-V-P-
R-A-K-I-H-S-L;

(SEQ ID NO: 27)

K-K-K-K-K-K-K-K-K-K-K-K-K-K-K-K-A-A-V-A-L-L-P-A-V-
L-L-A-L-L-A-P-M-S-V-L-T-P-L-L-L-R-G-L-T-G-S-A-R-R-
L-P-V-P-R-A-K-I-H-S-L;

(SEQ ID NO: 28)

K-K-K-K-A-A-V-A-L-L-P-A-V-L-L-A-L-L-A-P;

(SEQ ID NO: 29)

K-K-K-K-K-K-K-K-A-A-V-A-L-L-P-A-V-L-L-A-L-L-A-P;

(SEQ ID NO: 30)

K-K-K-K-K-K-K-K-K-K-K-K-A-A-V-A-L-L-P-A-V-L-L-A-L-
L-A-P;

(SEQ ID NO: 31)

K-K-K-K-K-K-K-K-K-K-K-K-K-K-K-K-A-A-V-A-L-L-P-A-V-
L-L-A-L-L-A-P;

(SEQ ID NO: 32)

Y-K-K-K-K-K-K-K-K-K-K-K-K-K-K-K-K-A-A-V-A-L-L-P-A-
V-L-L-A-L-L-A-P;

(SEQ ID NO: 33)

K-K-K-K-K-K-K-K-K-K-K-K-K-K-K-K-A-A-V-A-L-L-P-A-V-
L-L-A-L-L-A-P-A-A-V-A-L-L-P-A-V-L-L-A-L-L-A-P;

(SEQ ID NO: 34)

A-A-V-A-L-L-P-A-V-L-L-A-L-L-A-P-K-K-K-K-K-K-K-K-K-
K-K-K-A-A-V-A-L-L-P-A-V-L-L-A-L-L-A-P;

(SEQ ID NO: 35)

K-K-K-K-K-K-K-K-K-K-K-K-K-K-K-K-A-A-V-W-L-L-W-Y-V-
L-L-F-L-L-Y-L;
and

(SEQ ID NO: 36)

K-K-K-K-K-K-K-K-K-K-K-K-K-K-K-K-F-W-V-W-L-L-W-Y-V-
L-L-F-L-L-Y-L.

A carrier peptide, as described above, can be associated with a biologically active molecule. Examples of biologically active molecules include polypeptides; oligonucleotides; plasmids; small molecules; antibodies; antibody fragments; carbohydrates; lipids; glycolipids; antigens; and antigenic peptides. In some embodiments, oligonucleotides can be selected from coding DNA sequences; antisense DNA sequences; mRNAs, antisense RNA sequences; RNAis; and siRNAs. In other embodiments, a small molecule can be a therapeutic agent. In certain embodiments, the biologically active molecule is non-covalently bound to the carrier peptide.

Further provided herein is a method of transporting a biologically active molecule into a cell or mitochondrion of a subject (e.g., a human) comprising administering to the subject a complex comprising the molecule associated with a carrier peptide, as described herein.

In another embodiment, a method of transporting a biologically active molecule into a cell or mitochondrion is provided. Such a method comprises contacting the cell with a complex comprising a biologically active molecule associated with a carrier peptide, as described herein.

Also provided herein is a pharmaceutical composition comprising a carrier peptide. In certain embodiments the pharmaceutical composition contains a biologically active molecule associated with the carrier peptide. In some cases, the biologically active molecule is non-covalently bound to the carrier peptide.

In another aspect, a method of modulating expression of a nucleic acid of interest in a subject is provided, comprising administering to the subject a complex comprising a carrier peptide and an oligonucleotide. In some cases, the oligonucleotide is selected from coding DNA sequences; antisense DNA sequences; mRNAs, antisense RNA sequences; RNAis; and siRNAs. In certain embodiments, modulating results in decreased expression of a nucleic acid of interest, for example, when the nucleic acid of interest is an endogenous nucleic acid. In another embodiment, the nucleic acid is associated with cancer and the oligonucleotide is an SiRNA targeted to the endogenous nucleic acid. In other embodiments modulating results in increased expression of the nucleic acid of interest.

In a further aspect, a method of delivering a nucleic acid of interest into a cell is provided, the method comprising administering a complex comprising a carrier peptide and a nucleic acid of interest.

Provided herein is a kit comprising a carrier peptide. In some embodiments, the kit further comprises a biologically active molecule and/or instructions for administering to a subject. In another embodiment, the kit further comprises a label that that indicates that the contents are to be administered to a subject with a biologically active molecule.

The details of one or more embodiments are set forth in the accompanying drawings and the description below. Other features, objects, and advantages of the invention will be apparent from the description and drawings, and from the claims.

### DESCRIPTION OF DRAWINGS

FIG. 1 is an example of delivery of Alexa-IgG into mitochondria using SEQ ID NO:19. In the figure, blue corresponds to a stained nucleus; green is the color of the stained mitochondrion; red is the color of the Alexa-IgG complex; and orange is the result of the combination of red and green stain indicating that the Alexa-IgG complex has entered the mitochondria. 10 picomol of Alexa-tagged IgG were delivered with various concentrations of SEQ ID NO:19 (from 0 to 2000 pm).

FIG. 2 demonstrates delivery of FITC-tagged oligonucleotide with SEQ ID NO:19. The green color indicates mitochondria stained by the FITC-(SEQ ID NO:3) complex; blue color results from DAPI stain of the nucleus.

FIG. 3 illustrates delivery of a double-stranded circular plasmid (˜5.5 kb) into the mitochondria using SEQ ID NO:19. The blue color is the result of a DNA stain, while the small blue dots correspond to the mitochondria.

FIG. 4 displays the evaluation of the optimal length of the hydrophilic moiety (stretch of lysines, ‘K’s) for delivery of Alexa-IgG into TE85 cells. K4SP corresponds to SEQ ID NO:28; K8SP corresponds to SEQ ID NO:29; K12SP corresponds to SEQ ID NO:30; K16SP corresponds to SEQ ID NO:31.

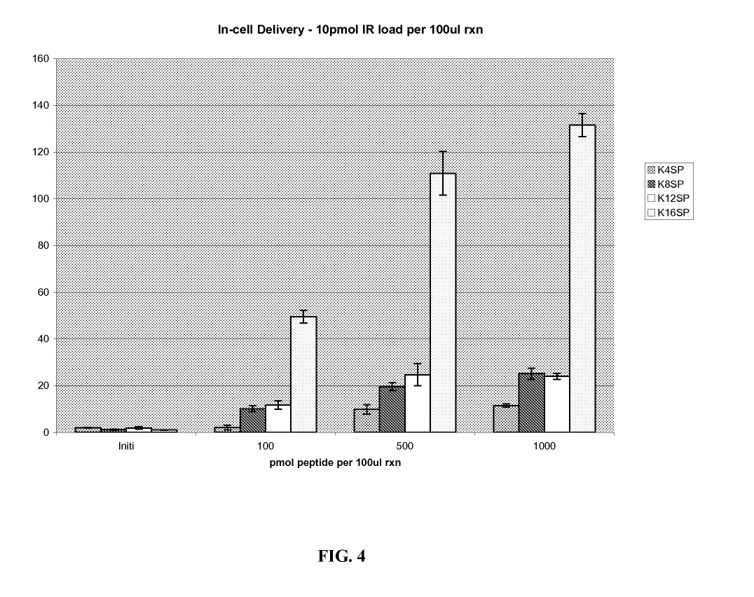

FIG. 5 details the evaluation of SP-mutants as delivery vehicles. YK16SP corresponds to SEQ ID NO:32; K16SP corresponds to SEQ ID NO:31; MODI-1 corresponds to SEQ ID NO:35; and MODI-2 corresponds to SEQ ID NO:36.

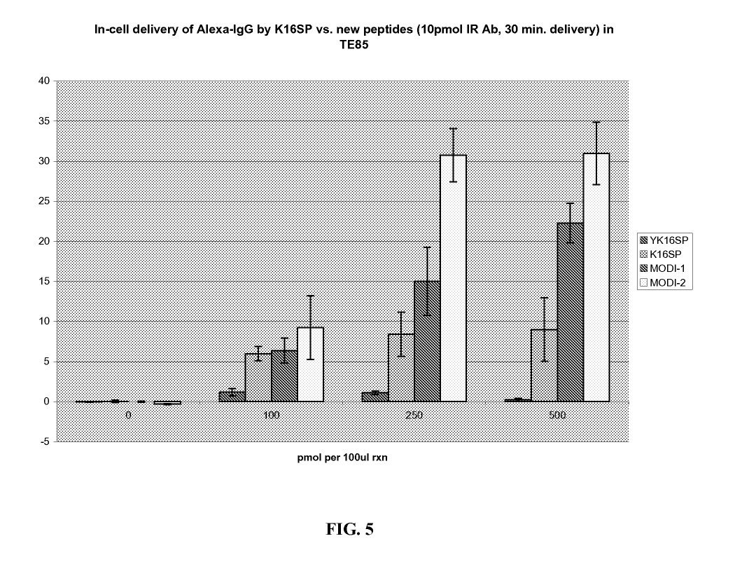

FIG. 6 illustrates the evaluation of various concatemers of K16 and SP sequences as protein (Alexa-IgG) delivery vehicles. K16SP corresponds to SEQ ID NO:32; SPK16SP corresponds to SEQ ID NO:345; and K16SPSP corresponds to SEQ ID NO:33.

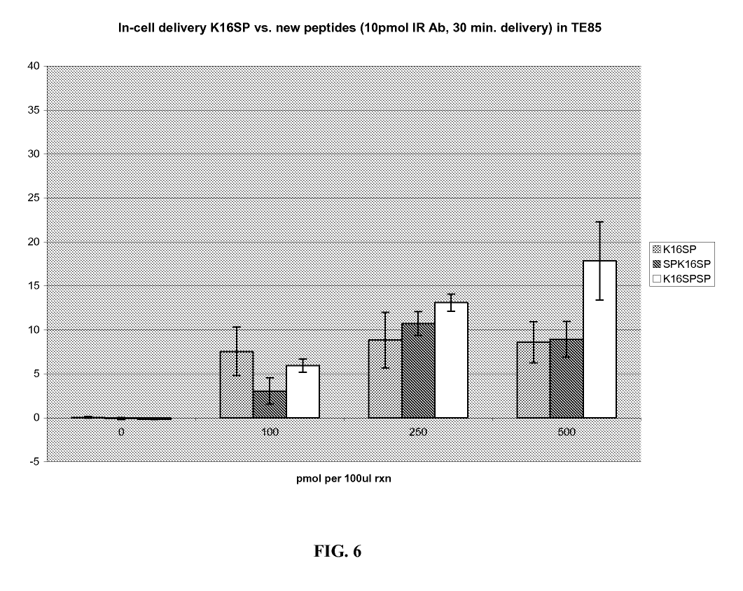

FIG. 7 displays expression of luciferase after transfection of 143B cells with a plasmid delivered by various peptides. K16SP corresponds to SEQ ID NO:31; K16SPSP corresponds to SEQ ID NO:33; MODI-1 corresponds to SEQ ID NO:35; MODI-2 corresponds to SEQ ID NO:36; and SPK16SP corresponds to SEQ ID NO:34.

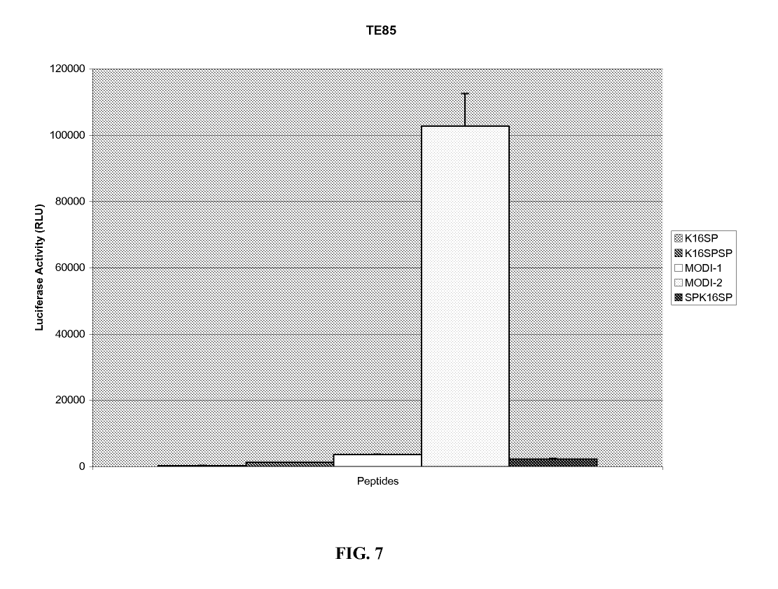

FIG. 8 displays FOB cytotoxicity of various carrier peptides. K16SP corresponds to SEQ ID NO:31; K16SPSP corresponds to SEQ ID NO:33; SPK16SP corresponds to SEQ ID NO:34; MODI-1 corresponds to SEQ ID NO:35; and MODI-2 corresponds to SEQ ID NO:36.

FIG. 9 illustrates TE85 cytotoxicity of various carrier peptides. K16SP corresponds to SEQ ID NO:31; K16SPSP corresponds to SEQ ID NO:34; SPK16SP corresponds to SEQ ID NO:33; MODI-1 corresponds to SEQ ID NO:35; and MODI-2 corresponds to SEQ ID NO:36.

FIG. 10 demonstrates the cytotoxicity of various carrier peptides after 72 hours in 143 B cells. K16SP corresponds to SEQ ID NO:31; K16SPSP corresponds to SEQ ID NO: 33; SPK16SP corresponds to SEQ ID NO:34; MODI-1 corresponds to SEQ ID NO:35; and MODI-2 corresponds to SEQ ID NO:36.

FIG. 11 displays delivery of Alex 568 (Rhodamine)-conjugated Goat anti-rabbit IgG into 143B cells using various carrier peptides (C (SEQ ID NO:18), CN (SEQ ID NO:21), KC (SEQ ID NO:31), and KNC (SEQ ID NO:21). The 568 nm fluorescence was converted to a green signal to enhance the contrast with DAPI (blue) at 362 nm.

### DETAILED DESCRIPTION

The peptides described herein can function as carrier peptides. These peptides can associate with (e.g., non-covalently bind) biologically active molecules to transport the biologically active molecules into cells and into cell organelles such as nuclei and mitochondria. In some cases, such transport may increase the therapeutic effectiveness of the biological molecules.

### I. Carrier Peptides

Provided herein is a carrier peptide selected from one of the following:

[T]ₐ-[PCS]_{b}-[N]_{c}-[PCS]_{d}-[SP]ₑ;
[T]ₐ-[PCS]_{b}-[SP]ₑ-[SP]ₑ-[PCS]_{d}-[N]_{c};
[N]_{c}-[PCS]_{b}-[SP]ₑ-[PCS]_{d}-[T]ₐ;
[N]_{c}-[PCS]_{b}-]T]ₐ-]PCS]_{d}-[SP]ₑ;
[SP]ₑ-[PCS]_{b}-[N]_{c}-[PCS]_{d}-[T]ₐ;
and
[SP]ₑ-[PCS]_{b}-[T]ₐ-[PCS]_{d}-[N]_{b};

wherein T is a transport peptide; PCS is a peptide cleavage sequence; N is a nuclear localization sequence; and SP is a signal peptide; wherein a is 1; b is 0 or 1; c is an integer from 0 to 5; d is 0 or 1; and e is an integer from 1 to 5.

A transport protein can be lysine or a non-natural lysine derivative, arginine or a non-natural arginine derivative, and combinations thereof. In some embodiments, the transport protein can be a sequence of four or more lysine residues or a sequence of 4 or more arginine residues. Non-limiting examples of transport proteins can include KKKK (SEQ ID NO:1); KKKKKKKK (SEQ ID NO:2); KKKKKKKKKKKK (SEQ ID NO:3); KKKKKKKKKKKKKKKK (SEQ ID NO:4); RRRR (SEQ ID NO:5); RRRRRRRR (SEQ ID NO:6); RRRRRRRRRRRR (SEQ ID NO:7); RRRRRRRRRRRRRRRR (SEQ ID NO:8); KRKR (SEQ ID NO:9); KKKR (SEQ ID NO:10); KKKRRRKKKRRR (SEQ ID NO:11); and KKKKRRRRKKKKRRRR (SEQ ID NO:12).

A peptide cleavage sequence (PCS) can be any suitable sequence that once in the cell can be enzymatically cleaved from the remaining sequence. In some embodiments, the PCS can include a furin protease cleavage recognition sequence. In other embodiments, the PCS is X-R-X-L-R-R-X (SEQ ID NO:13), wherein X is a hydrophobic amino acid (e.g., V, I, L, M, F, W, C, A, Y, H, T, S, P, or G).

A nuclear localization sequence (NLS) can be any sequence suitable for targeting the carrier peptide from the cytoplasm into the nucleus of the cell across the nuclear membrane. Any peptide, derivative thereof, or peptide analogue that functions to transport an associated molecule through a nuclear membrane can be used. Certain preferred specific NLSs include PKKKRKV (SEQ ID NO:14) which is a monopartite NLS from SV40 large T antigen, LVRKKRKTEEESPLKDKDAKKSKQE (SEQ ID NO:15) which is a bipartite NLS from SV40 N1 protein (Dingwall et al., Trends Biochem Sci (1991) 16(12):478-81), and PEVKKKRKPEYP (SEQ ID NO:16).

A signal peptide (SP) can be any sequence capable of translocating across the cell membrane into the interior of the selected target cell. In some cases, a SP is capable of translocating into the interior of an organelle (e.g., mitochondrion or nucleus). For example, a SP can comprise the sequence:

| | |
|---|---|
| X₁-X₂-V-X₃-L-L-X₄-X₅-V-L-L-X₆-L-L- | (SEQ ID NO: 17) |
| X₇-X₈ | |

wherein X₁-X₈ are independently L, A, W, F, Y, or V. In some embodiments, X₁ is A or F; X₂ is A or W; X₃ is A or W; X₄ is P or W; X₅ is A or Y; X₆ is A or F; X₇ is A or Y; X₈ is P or L. In some cases, the SP is A-A-V-A-L-L-P-A-V-L-L-A-L-L-A-P (SEQ ID NO:18). In another embodiment, the SP is A-A-V-A-L-L-P-A-V-L-L-A-L-L-A-P-M-S-V-L-T-P-L-L-L-R-G-L-T-G-S-A-R-R-L-P-V-P-R-A-K-I-H-S-L (SEQ ID NO:19).

In some embodiments, the carrier peptide, as described above, can be K-K-K-K-K-K-K-K-K-K-K-K-K-K-K-K-P-K-K-K-R-K-V-A-A-V-A-L-L-P-A-V-L-L-A-L-L-A-P (SEQ ID NO:20). In other embodiments, the carrier peptide can be A-A-V-A-L-L-P-A-V-L-L-A-L-L-A-P-P-K-K-K-R-K-V (SEQ ID NO:21).

In certain embodiments, a carrier peptide can comprise the sequence:

Kₙ-[X₁-X₂-V-X₃-L-L-X₄-X₅-V-L-L-X₆-L-L-X₇-X₈]ₘ

wherein X₁-X₈ are independently L, A, W, F, Y, or V; n is an integer from 4 to 20 (e.g., 4, 8, 12, 16, and 20); and m is an integer from 1 to 6 (e.g., 1, 2, 3, 4, 5, and 6). In specific embodiments, X₁ is A or F; X₂ is A or W; X₃ is A or W; X₄ is P or W; X₅ is A or Y; X₆ is A or F; X₇ is A or Y; X₈ is P or L. One example of the above carrier peptide K₂₀-[X₁-X₂-V-X₃-L-L-X₄-X₅-V-L-L-X₆-L-L-X₇-X₈]₆ (SEQ ID NO:22). Another way to describe the generic sequence above is Kₙ-[(SEQ ID NO:17)]ₘ, wherein n is an integer from 0 to 20; and m is an integer from 1 to 6.

In certain embodiments, the carrier peptide comprises the sequence:

[X₁-X₂-V-X₃-L-L-X₄-X₅-V-L-L-X₆-L-L-X₇-X₈]ₚ-Kₙ-[X₁-
X₂-V-X₃-L-L-X₄-X₅-V-L-L-X₆-L-L-X₇-X₈]ₘ

wherein X₁-X₈ are independently L, A, W, F, Y, or V; n is an integer from 0 to 20 (e.g., 4, 8, 12, 16, and 20); m is an integer from 1 to 6 (e.g., 1, 2, 3, 4, 5, and 6); and p is an integer from 1 to 6 (e.g., 1, 2, 3, 4, 5, and 6). In some embodiments, X₁ is A or F; X₂ is A or W; X₃ is A or W; X₄ is P or W; X₅ is A or Y; X₆ is A or F; X₇ is A or Y; X₈ is P or L. One example of the above carrier peptide is [X₁-X₂-V-X₃-L-L-X₄-X₅-V-L-L-X₆-L-L-X₇-X₈]₆-K₂₀-[X₁-X₂-V-X₃-L-L-X₄-X₅-V-L-L-X₆-L-L-X₇-X₈]₆ (SEQ ID NO:23). Another way to describe the generic sequence above is [(SEQ ID NO:17)]ₚ-Kₙ-[SEQ ID NO:17)]ₘ, wherein n is an integer from 0 to 20; m is an integer from 1 to 6; and p is an integer from 1 to 6.

In some embodiments, the carrier peptide is selected from the group consisting of:

(SEQ ID NO: 24)

K-K-K-K-A-A-V-A-L-L-P-A-V-L-L-A-L-L-A-P-M-S-V-L-T-
P-L-L-L-R-G-L-T-G-S-A-R-R-L-P-V-P-R-A-K-I-H-S-L;

(SEQ ID NO: 25)

K-K-K-K-K-K-K-K-A-A-V-A-L-L-P-A-V-L-L-A-L-L-A-P-M-
S-V-L-T-P-L-L-L-R-G-L-T-G-S-A-R-R-L-P-V-P-R-A-K-I-
H-S-L;

(SEQ ID NO: 26)

K-K-K-K-K-K-K-K-K-K-K-K-A-A-V-A-L-L-P-A-V-L-L-A-L-
L-A-P-M-S-V-L-T-P-L-L-L-R-G-L-T-G-S-A-R-R-L-P-V-P-
R-A-K-I-H-S-L;

(SEQ ID NO: 27)

K-K-K-K-K-K-K-K-K-K-K-K-K-K-K-K-A-A-V-A-L-L-P-A-V-
L-L-A-L-L-A-P-M-S-V-L-T-P-L-L-L-R-G-L-T-G-S-A-R-R-
L-P-V-P-R-A-K-I-H-S-L;

(SEQ ID NO: 28)

K-K-K-K-A-A-V-A-L-L-P-A-V-L-L-A-L-L-A-P;

(SEQ ID NO: 29)

K-K-K-K-K-K-K-K-A-A-V-A-L-L-P-A-V-L-L-A-L-L-A-P;

(SEQ ID NO: 30)

K-K-K-K-K-K-K-K-K-K-K-K-A-A-V-A-L-L-P-A-V-L-L-A-L-
L-A-P;

(SEQ ID NO: 31)

K-K-K-K-K-K-K-K-K-K-K-K-K-K-K-K-A-A-V-A-L-L-P-A-V-
L-L-A-L-L-A-P;

(SEQ ID NO: 32)

Y-K-K-K-K-K-K-K-K-K-K-K-K-K-K-K-K-A-A-V-A-L-L-P-A-
V-L-L-A-L-L-A-P;

(SEQ ID NO: 33)

K-K-K-K-K-K-K-K-K-K-K-K-K-K-K-K-A-A-V-A-L-L-P-A-V-
L-L-A-L-L-A-P-A-A-V-A-L-L-P-A-V-L-L-A-L-L-A-P;

(SEQ ID NO: 34)

A-A-V-A-L-L-P-A-V-L-L-A-L-L-A-P-K-K-K-K-K-K-K-K-K-
K-K-K-A-A-V-A-L-L-P-A-V-L-L-A-L-L-A-P;

(SEQ ID NO: 35)

K-K-K-K-K-K-K-K-K-K-K-K-K-K-K-K-A-A-V-W-L-L-W-Y-V-
L-L-F-L-L-Y-L;
and

(SEQ ID NO: 36)

K-K-K-K-K-K-K-K-K-K-K-K-K-K-K-K-F-W-V-W-L-L-W-Y-V-
L-L-F-L-L-Y-L.

The peptides disclosed herein are described using the standard one letter amino acid abbreviations. The amino acids can be in their D or L form. All peptides can be prepared using methods known to those having ordinary skill in the art, including solid phase methods.

Further provided herein are complexes comprising a biologically active molecule associated with a carrier peptide. In some embodiments, the biologically active molecule is non-covalently bound to the carrier peptide. A carrier peptide can comprise any sequence as described previously.

As used herein, a “biologically active molecule” includes any molecule which, if imported into a cell, can have a biological effect. Examples of biologically active molecules include polypeptides, which include functional domains of biologically active molecules, particular examples include growth factors, enzymes, transcription factors, toxins, antigenic peptides (as for vaccines), antibodies, and antibody fragments. Additional examples of biologically active molecules include oligonucleotides, such as natural or engineered plasmids, coding DNA sequences, antisense DNA sequences, mRNAs, antisense RNA sequences, RNAis, and siRNAs; carbohydrates; lipids; and glycolipids. Further examples of biologically active molecules include small molecules, including therapeutic agents, in particular those with a low cell or mitochondrion membrane permeability. Some examples of these therapeutic agents include cancer drugs, such as Daunorubicin and toxic chemicals which, because of the lower dosage that can be administered by this method, can now be more safely administered.

Yet another example of a biologically active molecule is an antigenic peptide. Antigenic peptides can be administered to provide immunological protection when imported by cells involved in the immune response. Other examples include immunosuppressive peptides (e.g., peptides that block autoreactive T cells, which peptides are known in the art). Numerous other examples of biologically active molecules will be apparent to the skilled artisan.

Polypeptides from a few amino acids to around a thousand amino acids can be used. In some embodiments, the size range for polypeptides is from a few amino acids to about 250 amino acids (e.g., about 3 to about 250 amino acids; about 20 to about 250 amino acids; about 50 to about 250 amino acids; about 100 to about 250 amino acids; about 150 to about 250 amino acids; about 3 amino acids to about 200 amino acids; about 3 amino acids to about 150 amino acids; about 3 amino acids to about 175 amino acids; about 3 amino acids to about 125 amino acids; about 25 amino acids to about 200 amino acids; about 50 amino acids to about 150 amino acids; and about 75 amino acids to about 225 amino acids). For any molecule, size ranges can be up to about a molecular weight of about 1 million. In some embodiments, the size ranges up to a molecular weight of about 25,000, and in particular embodiments, the size ranges can be up to a molecular weight of about 3,000.

By “antisense” it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004; Agrawal et al., U.S. Pat. No. 5,591,721; Agrawal, U.S. Pat. No. 5,652,356). Typically, antisense molecules will be complementary to a target sequence along a single contiguous sequence of the antisense molecule. However, in certain embodiments, an antisense molecule may bind to a substrate such that the substrate molecule forms a loop, and/or an antisense molecule may bind such that the antisense molecule forms a loop. Thus, the antisense molecule may be complementary to two (or even more) non-contiguous substrate sequences or two (or even more) non-contiguous sequence portions of an antisense molecule may be complementary to a target sequence or both.

RNA interference (RNAi) and short intervening RNA (siRNA) sequences can be used to modulate (e.g., inhibit) gene expression (see, e.g., Elbashir et al., 2001, Nature, 411, 494 498; and Bass, 2001, Nature, 411, 428 429; Bass, 2001, Nature, 411, 428 429; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914). In one embodiment, a siRNA molecule comprises a double stranded RNA wherein one strand of the RNA is complimentary to the RNA of interest. In another embodiment, a siRNA molecule comprises a double stranded RNA wherein one strand of the RNA comprises a portion of a sequence of an RNA of interest. In yet another embodiment, a siRNA molecule of the invention comprises a double stranded RNA wherein both strands of RNA are connected by a non-nucleotide linker Alternately, a siRNA molecule of the invention comprises a double stranded RNA wherein both strands of RNA are connected by a nucleotide linker, such as a loop or stem loop structure.

The term “antibody” as used herein refers to immunoglobulin molecules and immunologically active portions of immunoglobulin molecules (i.e., molecules that contain an antigen binding site that specifically binds to a peptide). An antibody can be a monoclonal antibody, a polyclonal antibody, a humanized antibody, a fully human antibody, a single chain antibody, a chimeric antibody, or a fragment thereof. The term “antibody fragment” of a full length antibody refers to one or more fragments of a full-length antibody that retain the ability to specifically bind to a target of interest.

“Associated with” as used herein is meant that the biologically active molecule is conjugated to the carrier peptide in such a manner that when the carrier peptide crosses the cell membrane, the molecule is also imported across the cell membrane. In certain embodiments, the biologically active molecule is non-covalently bound to the carrier peptide. In other embodiments, the carrier peptide may be covalently bound, either directly or indirectly (e.g., through a linker), to the biologically active molecule.

A linker can be any moiety suitable for linking a carrier peptide to a biologically active molecule. A linker can be bound at the C-terminus, the N-terminus, or both, of a carrier peptide. Additionally, a linker can be bound to the side chain of a carrier peptide. If a carrier peptide is bound to multiple linkers, each linker can be different. A linker can be covalently linked to a side chain of an amino acid, e.g., lysine, glutamine, cysteine, methionine, glutamate, aspartate, asparagine.

In some embodiments an amino acid side chain can serve as the linker. For example the epsilon amino group (ε-NH₂) can be used to conjugate to a carrier for instance through an amide or thiourea linkage. Similarly the delta amino group of ornithine (orn), the gamma amino group of diaminobutyric acid (dab), or the beta amino group of diamino proprionic acid (dpr) can also act as linkers. These amino acids may be at the C- or N-terminus of the carrier peptide or they may be positioned within the carrier peptide sequence.

The complex composed of a biologically active molecule and a carrier peptide can be prepared by any method known by those having ordinary skill in the art. In some embodiments, the carrier peptide and the biologically active molecule are combined, incubated at room temperature, and then used (see, e.g., Example 1).

### II. Methods of Use

Also provided herein are methods of using carrier peptides to transport a biologically active molecule into a cell of a subject. In some embodiments, a carrier peptide can be used to transport a biologically active molecule into a mitochondrion of a subject.

A subject can include both mammals and non-mammals. Mammals include, for example, humans; nonhuman primates, e.g. apes and monkeys; cattle; horses; sheep; rats; mice; pigs; and goats. Non-mammals include, for example, fish and birds.

A carrier peptide can comprise any sequence described previously. For example, a carrier peptide can comprise the sequence:

Kₙ-[X₁-X₂-V-X₃-L-L-X₄-X₅-V-L-L-X₆-L-L-X₇-X₈]ₘ

wherein X₁ is A or F; X₂ is A or W; X₃ is A or W; X₄ is P or W; X₅ is A or Y; X₆ is A or F; X₇ is A or Y; X₈ is P or L; n is an integer from 4 to 20; and m is an integer from 1 to 6.

In certain embodiments, the carrier peptide comprises the sequence:

[X₁-X₂-V-X₃-L-L-X₄-X₅-V-L-L-X₆-L-L-X₇-X₈]ₚ-Kₙ-[X₁-
X₂-V-X₃-L-L-X₄-X₅-V-L-L-X₆-L-L-X₇-X₈]ₘ

wherein X₁ is A or F; X₂ is A or W; X₃ is A or W; X₄ is P or W; X₅ is A or Y; X₆ is A or F; X₇ is A or Y; X₈ is P or L; n is an integer from 0 to 20; m is an integer from 1 to 6; and p is an integer from 1 to 6.

In some embodiments, the carrier peptide is selected from the group consisting of:

(SEQ ID NO: 24)

K-K-K-K-A-A-V-A-L-L-P-A-V-L-L-A-L-L-A-P-M-S-V-L-T-
P-L-L-L-R-G-L-T-G-S-A-R-R-L-P-V-P-R-A-K-I-H-S-L;

(SEQ ID NO: 25)

K-K-K-K-K-K-K-K-A-A-V-A-L-L-P-A-V-L-L-A-L-L-A-P-M-
S-V-L-T-P-L-L-L-R-G-L-T-G-S-A-R-R-L-P-V-P-R-A-K-I-
H-S-L;

(SEQ ID NO: 26)

K-K-K-K-K-K-K-K-K-K-K-K-A-A-V-A-L-L-P-A-V-L-L-A-L-
L-A-P-M-S-V-L-T-P-L-L-L-R-G-L-T-G-S-A-R-R-L-P-V-P-
R-A-K-I-H-S-L;

(SEQ ID NO: 27)

K-K-K-K-K-K-K-K-K-K-K-K-K-K-K-K-A-A-V-A-L-L-P-A-V-
L-L-A-L-L-A-P-M-S-V-L-T-P-L-L-L-R-G-L-T-G-S-A-R-R-
L-P-V-P-R-A-K-I-H-S-L;

(SEQ ID NO: 28)

K-K-K-K-A-A-V-A-L-L-P-A-V-L-L-A-L-L-A-P;

(SEQ ID NO: 29)

K-K-K-K-K-K-K-K-A-A-V-A-L-L-P-A-V-L-L-A-L-L-A-P;

(SEQ ID NO: 30)

K-K-K-K-K-K-K-K-K-K-K-K-A-A-V-A-L-L-P-A-V-L-L-A-L-
L-A-P;

(SEQ ID NO: 31)

K-K-K-K-K-K-K-K-K-K-K-K-K-K-K-K-A-A-V-A-L-L-P-A-V-
L-L-A-L-L-A-P;

(SEQ ID NO: 32)

Y-K-K-K-K-K-K-K-K-K-K-K-K-K-K-K-K-A-A-V-A-L-L-P-A-
V-L-L-A-L-L-A-P;

(SEQ ID NO: 33)

K-K-K-K-K-K-K-K-K-K-K-K-K-K-K-K-A-A-V-A-L-L-P-A-V-
L-L-A-L-L-A-P-A-A-V-A-L-L-P-A-V-L-L-A-L-L-A-P;

(SEQ ID NO: 34)

A-A-V-A-L-L-P-A-V-L-L-A-L-L-A-P-K-K-K-K-K-K-K-K-K-
K-K-K-A-A-V-A-L-L-P-A-V-L-L-A-L-L-A-P;

(SEQ ID NO: 35)

K-K-K-K-K-K-K-K-K-K-K-K-K-K-K-K-A-A-V-W-L-L-W-Y-V-
L-L-F-L-L-Y-L;
and

(SEQ ID NO: 36)

K-K-K-K-K-K-K-K-K-K-K-K-K-K-K-K-F-W-V-W-L-L-W-Y-V-
L-L-F-L-L-Y-L.

Transporting a biologically active molecule can include importing the molecule across the membrane of the cell, mitochondrion, or nucleus.

Included in the method of transporting a biologically active molecule into a cell or membrane is administering to a subject a complex comprising a biologically active molecule associated with a carrier peptide.

The complex can be administered by any route, e.g., IV, intramuscular, SC, oral, intranasal, inhalation, transdermal, and parenteral.

The complex can be formulated with a pharmaceutically acceptable carrier selected on the basis of the selected route of administration and standard pharmaceutical practice. The complex may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa. Suitable dosage forms may comprise, for example, tablets, capsules, solutions, parenteral solutions, troches, suppositories, or suspensions.

For parenteral administration, the complex may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the complex. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or non-aqueous solution, dispersion, suspension or emulsion.

For oral administration, the complex may be combined with one or more solid inactive ingredients for the preparation of tablets, capsules, pills, powders, granules or other suitable oral dosage forms. For example, the complex may be combined with at least one excipient such as fillers, binders, humectants, disintegrating agents, solution retarders, absorption accelerators, wetting agents absorbents or lubricating agents.

The specific dose of a complex will, of course, be determined by the particular circumstances of the individual patient including the size, weight, age and sex of the patient, the nature and stage of the disease being treated, the aggressiveness of the disease disorder, and the route of administration of the compound.

In a further embodiment, a carrier peptide can be used to transport a biologically active molecule into a cell or mitochondrion in vitro. In certain embodiments, the method can include contacting the cell with a complex of the carrier peptide associated with the biologically active molecule. In some embodiments, the carrier peptide is non-covalently bound to the biologically active molecule.

As used herein, “contacting” can include any method known in the art that facilitates the carrier peptide and biologically active molecule crossing the membrane of the cell or mitochondrion.

Provided herein is a method of treating a disease in a subject in need of such treatment by administering to the subject a complex comprising a carrier peptide, as described previously, and a biologically active molecule. In certain embodiments, the biologically active molecule is a therapeutic agent.

Examples of diseases that may be treated by this method include cancer, Leber's optic hereditary neuropathy (LOHN), myoclonic epilepsy and ragged red fiber (MERRF), and Alzheimer's.

In another embodiment, a method of modulating expression of a nucleic acid of interest is disclosed herein. This method includes administering a complex comprising a carrier peptide and a biologically active molecule to a subject. In some embodiments, the biologically active molecule can be an oligonucleotide as described previously. In certain embodiments, the oligonucleotide can be an endogenous nucleic acid, while in other embodiments, the oligonucleotide can be an exogenous nucleic acid.

As used herein, “modulating” means increasing or decreasing the expression of a nucleic acid of interest. For example, a nucleic acid encoding a polypeptide of interest, when transported into a cell or mitochondrion, can increase the expression of the polypeptide in the subject. In one embodiment, expression of a polypeptide of interest can inhibit one or more functions of an endogenous polypeptide. For example, a nucleic acid that encodes an inhibitory polypeptide can be delivered to the cell or mitochondrion to result in inhibition of the function of an endogenous polypeptide. In another embodiment, a nucleic acid can inhibit expression of an endogenous polypeptide (e.g., an oncogenic peptide). A number of nucleic acid based methods, including antisense RNA, ribozyme directed RNA cleavage, post-transcriptional gene silencing (PTGS), e.g., RNAi and siRNA), and transcriptional gene silencing (TGS) are known to inhibit endogenous gene expression. In one example, an siRNA, targeted for an endogenous nucleic acid of interest, can be associated with a carrier peptide as described herein. Once in the cell or mitochondrion, the siRNA can function to inhibit expression of an endogenous polypeptide.

Transportation of oligonucleotides into cells and mitochondria can be utilized as a therapeutic treatment for diseases and disorders responsive to such therapy (e.g., gene therapy). In certain embodiments, such therapy can include treatment of cancer.

### III. Pharmaceutical Compositions

Provided herein are pharmaceutical compositions comprising a biologically active molecule associated with a carrier peptide. In some embodiments, the biologically active molecule is non-covalently bound to the carrier peptide.

The pharmaceutical compositions provided herein contain a biologically active molecule associated with a carrier peptide, in an amount that results in transportation of the biologically active molecule into the cell or mitochondrion, and a pharmaceutically acceptable carrier. Pharmaceutical carriers suitable for administration of the compounds provided herein include any such carriers known to those skilled in the art to be suitable for the particular mode of administration.

The compositions can be, in one embodiment, formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation, dry powder inhalers, and ointments (see, e.g., Ansel Introduction to Pharmaceutical Dosage Forms, Fourth Edition 1985, 126).

The concentration of the biologically active molecule associated with a carrier peptide in the pharmaceutical composition will depend on absorption, inactivation and excretion rates of the compounds, the physicochemical characteristics of the compounds, the dosage schedule, and amount administered as well as other factors known to those of skill in the art.

The pharmaceutical composition may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing a biologically active molecule associated with a carrier peptide in the range of 0.005% to 100% with the balance made up with a non-toxic carrier may be prepared. Methods for preparation of these compositions are known to those skilled in the art. The contemplated compositions may contain 0.001%-100% active ingredient, in one embodiment 0.1-95%, in another embodiment 75-85%.

### IV. Kits

Also provided herein are kits. Typically, a kit includes a carrier peptide, as described previously. In some embodiments, a kit includes a carrier peptide and a biologically active molecule. In certain embodiments, a kit can include one or more delivery systems, e.g., for a biologically active molecule, carrier peptide, or any combination thereof, and directions for use of the kit (e.g., instructions for administering to a subject). In certain embodiments, a kit can include a biologically active molecule, a carrier peptide, and/or a complex of a biologically active molecule and a carrier peptide. In some embodiments, the kit can include a carrier peptide and a label that indicates that the contents are to be administered to a subject with a biologically active molecule.

### EXAMPLES

### General Methods

All peptides were synthesized using solid phase synthesis. Chariot (a commercially-available peptide reagent for non-covalent delivery of proteins into cells) and beta-galactosidase were purchased from Active Motif (Carlsbad, Calif.). Alexa Fluor 568 goat anti-rabbit IgG and EGFP proteins were from Invitrogen (Eugene, Oreg.) and Biovision (Mountain View, Calif.), respectively. IRDye 800-conjugated affinity-purified anti-mouse IgG (goat) was purchased from Rockland (Gilbertsville, Pa.). Antibody against alpha-tubulin was purchased from Santa Cruz Biotechnology (www.scbt.com).

Adherent 143B, TE85, MG63 and FOB cell lines were cultured in Dulbecco's modified eagle's medium (DMEM) supplemented with 1% (vol/vol) 200 mM glutamine, 1% (vol/vol) antibiotics (streptomycin, 10,000 μg/ml; penicillin, 10,000 IU/ml), and 10% (wt/vol) FBS, at 37° C. in a humidified atmosphere containing 5% CO₂.

### Example 1

### Binding of Carrier Peptides with Protein

Binding experiments were performed using Xenobind™ plates (Xenopore). Peptide solution was made in NaOH at pH 13 (which is above the isoeletric point of the peptide). The protocol supplied by the manufacturer was essentially followed for anchoring peptides onto the plates and subsequent binding of IR-IgG (Invitrogen) with the anchored peptides. Briefly, each well of a Xenobind™ plate was filled with 200 μl of peptide solution. The plate was incubated overnight at room temperature. Peptide solutions were removed from wells followed by washing with PBST (containing 0.1% TWEEN 20). IR-IgG in OptiMem® was added to each well and incubated at 37° C. for 30 min. The plate was then finally washed with PBST and the IR-intensity was measured with Odyssey Infrared Imaging System (LI-COR).

### Example 2

### Peptide-Mediated Protein Delivery

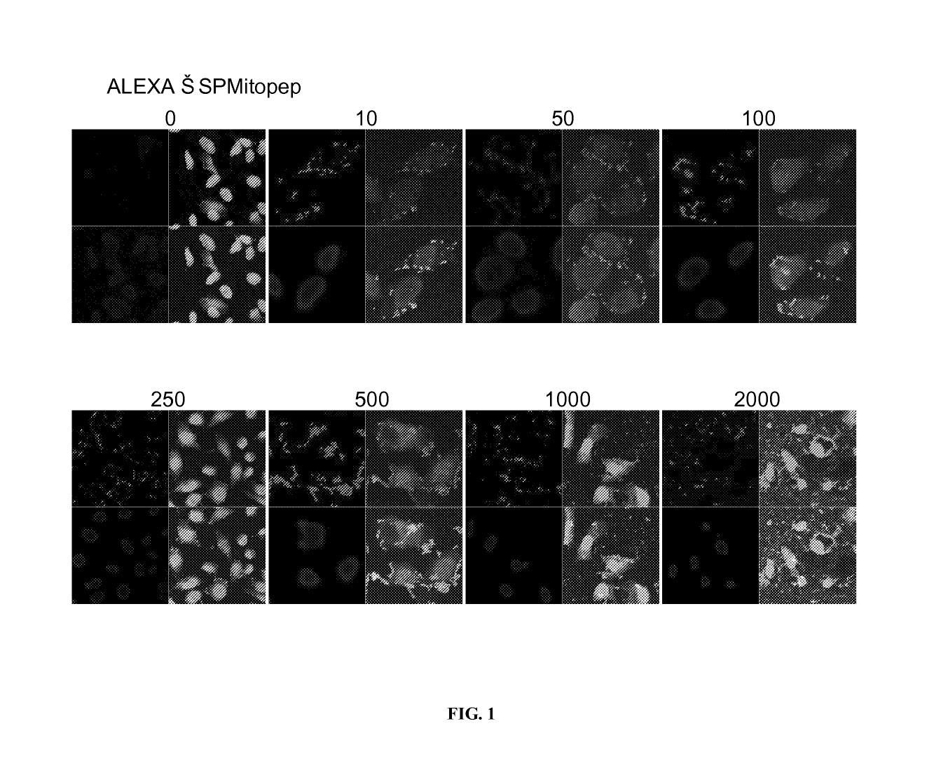

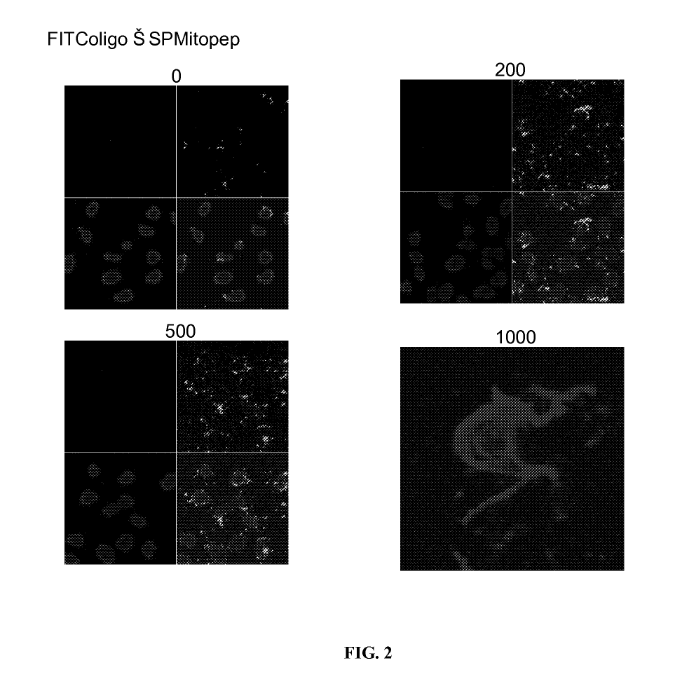

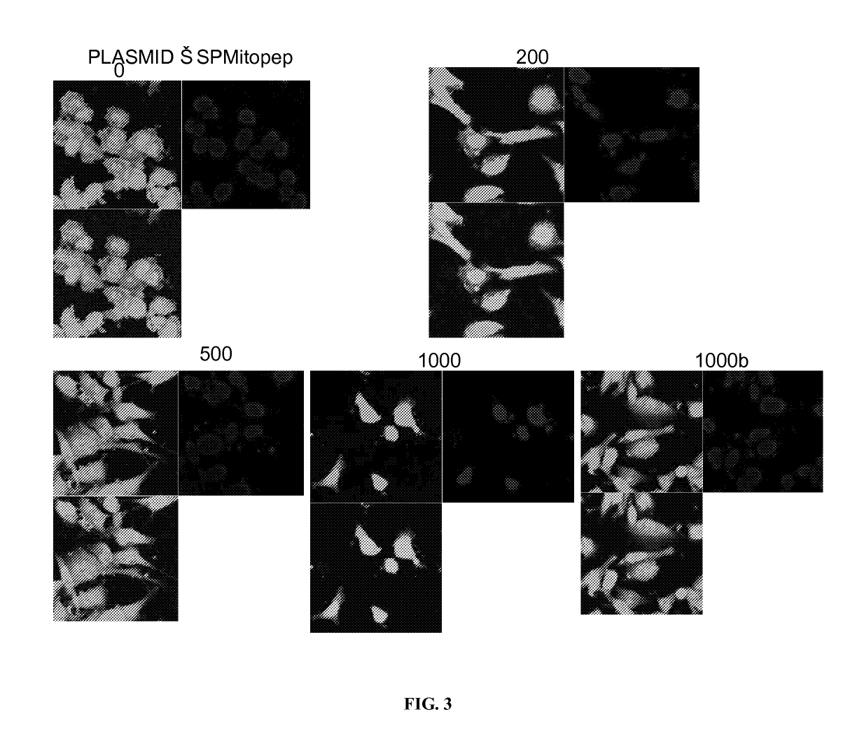

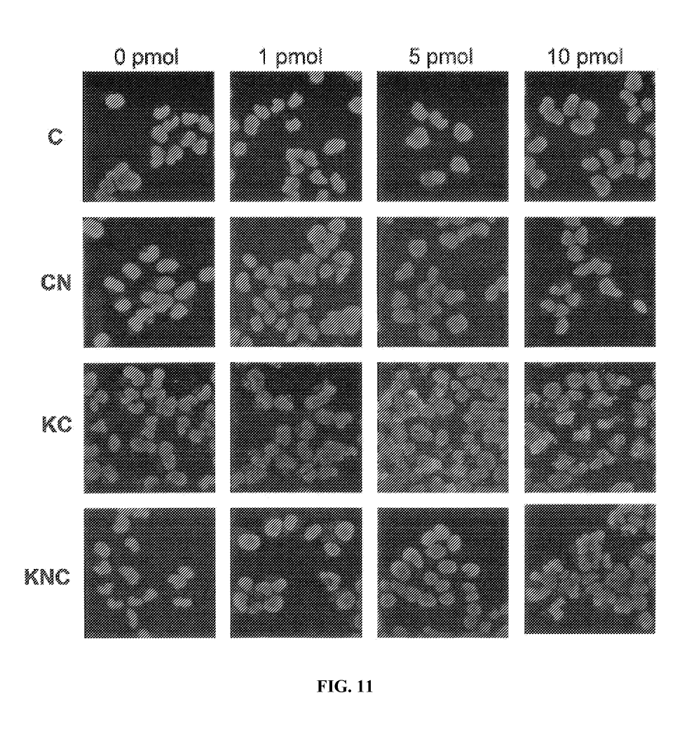

Cells, indicated above, were grown in 8-well culture slides. Peptide/protein complexes were formed in a volume of 100 μl in OptiMEM and incubated for 30 min at 37° C. Approximately 10,000 Cells grown to 75% confluence were overlaid with these preformed complexes. After 30 min of incubation at 37° C., 500 μl of fresh DMEM supplemented with 10% FBS was added to the cells and incubated for an additional 30 min. Cells were then washed with PBS followed by fixing with 4% paraformaldehyde in PBS. Cells were then mounted with flourescent-FragEL Mounting media (Calbiochem) containing DAPI nuclear stain before microscopic analysis. See, e.g., FIGS. 1, 2, 3, and 11.

### Example 3

### Cytotoxicity Assay

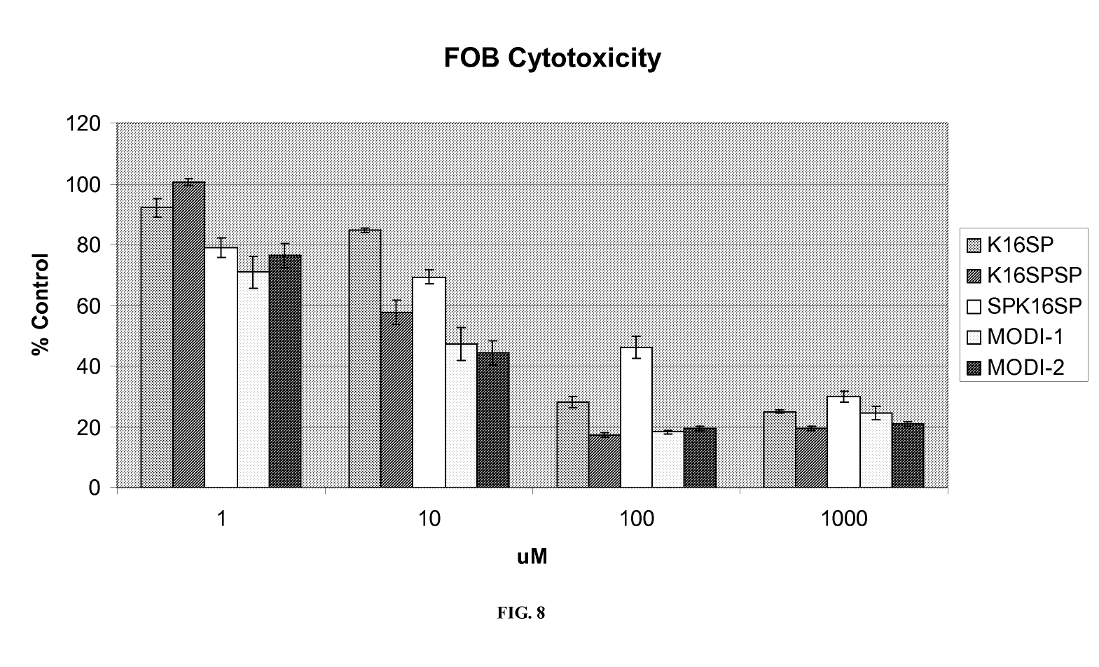

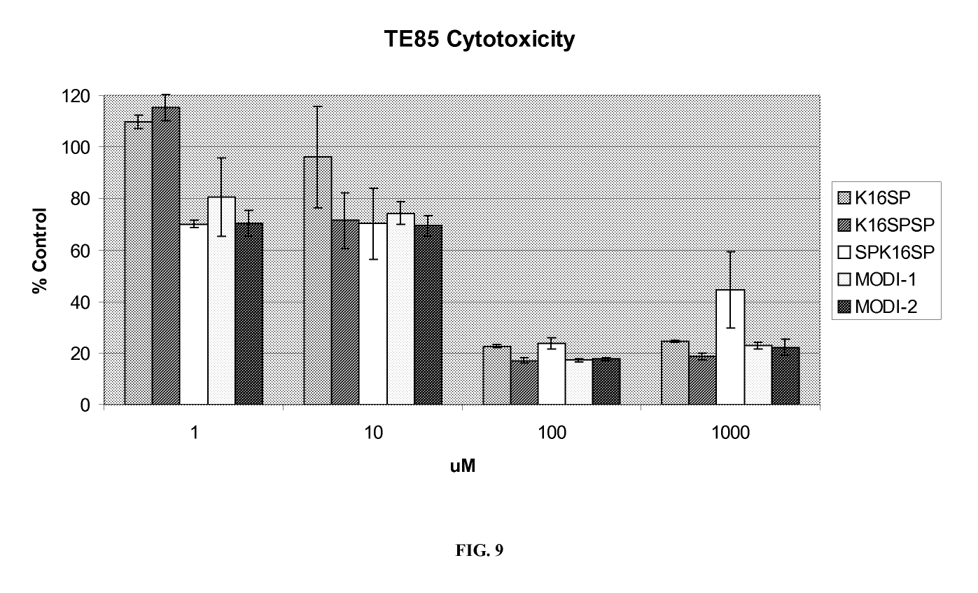

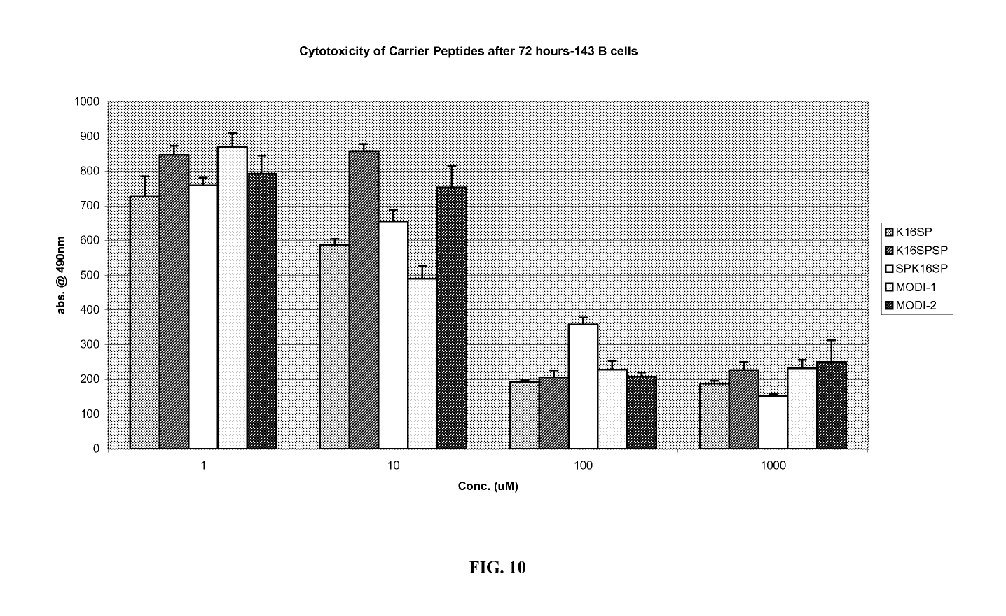

The cytotoxicity of various peptides were investigated in different cell lines (143B, TE85, FOB). Cells were incubated with 1.0-1000 μM peptide in DMEM supplemented with 10% FBS in 96-well plates (10⁴ cells per well). Cytotoxicity was evaluated in a colorimetric assay using 3-(4,5-dimethylthiazole-2-yl)-2,5-diphenyltetrazolium bromide (MTT) after 3 days of incubation (MTT assay kit from Promega, Madison, Wis.). See, e.g., FIGS. 8-10.

A number of embodiments have been described. Nevertheless, it will be understood that various modifications may be made without departing from the spirit and scope of the invention. Accordingly, other embodiments are within the scope of the following claims.

1. A carrier peptide comprising the sequence:
(SEQ ID NO: 37)
Kₙ-[X₁-X₂-V-X₃-L-L-X₄-X₅-V-L-L-X₆-L-L-X₇-X₈]ₘ
wherein:
X₁-X₈ are independently L, A, W, F, Y, or V;
n is an integer from 4 to 20; and
m is an integer from 1 to 6.

2. The carrier peptide of claim 0, wherein the carrier peptide comprises the sequence:
(SEQ ID NO: 38)
[X₁-X₂-V-X₃-L-L-X₄-X₅-V-L-L-X₆-L-L-X₇-X₈]ₚ-Kₙ-[X₁-
X₂-V-X₃-L-L-X₄-X₅-V-L-L-X₆-L-L-X₇-X₈]ₘ
wherein:
X₁-X₈ are independently L, A, W, F, Y, or V;
n is an integer from 0 to 20;
m is an integer from 1 to 6; and
p is an integer from 1 to 6.

3. The carrier peptide of claim 0 or 0, wherein:
X₁ is A or F;
X₂ is A or W;
X₃ is A or W;
X₄ is P or W;
X₅ is A or Y;
X₆ is A or F;
X₇ is A or Y;
X₈ is P or L.

4. The carrier peptide of claim 0, wherein the carrier peptide is selected from the group consisting of:
(SEQ ID NO: 24)
K-K-K-K-A-A-V-A-L-L-P-A-V-L-L-A-L-L-A-P-M-S-V-L-T-
P-L-L-L-R-G-L-T-G-S-A-R-R-L-P-V-P-R-A-K-I-H-S-L;
(SEQ ID NO: 25)
K-K-K-K-K-K-K-K-A-A-V-A-L-L-P-A-V-L-L-A-L-L-A-P-M-
S-V-L-T-P-L-L-L-R-G-L-T-G-S-A-R-R-L-P-V-P-R-A-K-I-
H-S-L;
(SEQ ID NO: 26)
K-K-K-K-K-K-K-K-K-K-K-K-A-A-V-A-L-L-P-A-V-L-L-A-L-
L-A-P-M-S-V-L-T-P-L-L-L-R-G-L-T-G-S-A-R-R-L-P-V-P-
R-A-K-I-H-S-L;
(SEQ ID NO: 27)
K-K-K-K-K-K-K-K-K-K-K-K-K-K-K-K-A-A-V-A-L-L-P-A-V-
L-L-A-L-L-A-P-M-S-V-L-T-P-L-L-L-R-G-L-T-G-S-A-R-R-
L-P-V-P-R-A-K-I-H-S-L;
(SEQ ID NO: 28)
K-K-K-K-A-A-V-A-L-L-P-A-V-L-L-A-L-L-A-P;
(SEQ ID NO: 29)
K-K-K-K-K-K-K-K-A-A-V-A-L-L-P-A-V-L-L-A-L-L-A-P;
(SEQ ID NO: 30)
K-K-K-K-K-K-K-K-K-K-K-K-A-A-V-A-L-L-P-A-V-L-L-A-L-
L-A-P;
(SEQ ID NO: 31)
K-K-K-K-K-K-K-K-K-K-K-K-K-K-K-K-A-A-V-A-L-L-P-A-V-
L-L-A-L-L-A-P;
(SEQ ID NO: 32)
Y-K-K-K-K-K-K-K-K-K-K-K-K-K-K-K-K-A-A-V-A-L-L-P-A-
V-L-L-A-L-L-A-P;
(SEQ ID NO: 33)
K-K-K-K-K-K-K-K-K-K-K-K-K-K-K-K-A-A-V-A-L-L-P-A-V-
L-L-A-L-L-A-P-A-A-V-A-L-L-P-A-V-L-L-A-L-L-A-P;
(SEQ ID NO: 34)
A-A-V-A-L-L-P-A-V-L-L-A-L-L-A-P-K-K-K-K-K-K-K-K-K-
K-K-K-A-A-V-A-L-L-P-A-V-L-L-A-L-L-A-P;
(SEQ ID NO: 35)
K-K-K-K-K-K-K-K-K-K-K-K-K-K-K-K-A-A-V-W-L-L-W-Y-V-
L-L-F-L-L-Y-L;
and
(SEQ ID NO: 36)
K-K-K-K-K-K-K-K-K-K-K-K-K-K-K-K-F-W-V-W-L-L-W-Y-V-
L-L-F-L-L-Y-L.

5. The carrier peptide of claim 0, wherein the carrier peptide is associated with a biologically active molecule.

6. The carrier peptide of claim 0, wherein the biologically active molecule is selected from the group consisting of: polypeptides; oligonucleotides; plasmids; small molecules; antibodies; antibody fragments; carbohydrates; lipids; glycolipids; antigens; and antigenic peptides.

7. The carrier peptide of claim 0, wherein the biologically active molecule is non-covalently bound to the carrier peptide.

8. A complex comprising a biologically active molecule associated with a carrier peptide; wherein the carrier peptide comprises the sequence:
(SEQ ID NO: 37)
Kₙ-[X₁-X₂-V-X₃-L-L-X₄-X₅-V-L-L-X₆-L-L-X₇-X₈]ₘ
wherein:
X₁-X₈ are independently L, A, W, F, Y, or V;
n is an integer from 4 to 20; and
m is an integer from 1 to 6.

9. The complex of claim 0, wherein:
X₁ is A or F;
X₂ is A or W;
X₃ is A or W;
X₄ is P or W;
X₅ is A or Y;
X₆ is A or F;
X₇ is A or Y;
X₈ is P or L.

10. The complex of claim 0, wherein the biologically active molecule is selected from the group consisting of: polypeptides; oligonucleotides; plasmids; small molecules; antibodies; antibody fragments; carbohydrates; lipids; glycolipids; antigens; and antigenic peptides.

11. The complex of claim 0, wherein the biologically active molecule is non-covalently bound to the carrier peptide.

12. A method of transporting a biologically active molecule into a cell of a subject comprising administering to the subject a complex comprising the molecule associated with a carrier peptide; wherein the carrier peptide comprises the sequence:
(SEQ ID NO: 41)
Kₙ-[X₁-X₂-V-X₃-L-L-X₄-X₅-V-L-L-X₆-L-L-X₇-X₈]ₘ
wherein:
X₁-X₈ are independently L, A, W, F, Y, or V;
n is an integer from 0 to 20; and
m is an integer from 1 to 6;
thereby transporting the molecule into the cell.

13. The method of claim 0, wherein the carrier peptide comprises the sequence:
(SEQ ID NO: 38)
[X₁-X₂-V-X₃-L-L-X₄-X₅-V-L-L-X₆-L-L-X₇-X₈-]ₚKₙ[X₁-
X₂-V-X₃-L-L-X₄-X₅-V-L-L-X₆-L-L-X₇-X₈]ₘ
wherein:
X₁-X₈ are independently L, A, W, F, Y, or V;
n is an integer from 0 to 20;
m is an integer from 1 to 6; and
p is an integer from 1 to 6.

14. The method of claim 0 or 0, wherein:
X₁ is A or F;
X₂ is A or W;
X₃ is A or W;
X₄ is P or W;
X₅ is A or Y;
X₆ is A or F;
X₇ is A or Y;
X₈ is P or L.

15. The method of claim 0, wherein the carrier peptide is selected from the group consisting of:
(SEQ ID NO: 24)
K-K-K-K-A-A-V-A-L-L-P-A-V-L-L-A-L-L-A-P-M-S-V-L-
T-P-L-L-L-R-G-L-T-G-S-A-R-R-L-P-V-P-R-A-K-I-H-S-
L;
(SEQ ID NO: 25)
K-K-K-K-K-K-K-K-A-A-V-A-L-L-P-A-V-L-L-A-L-L-A-P-
M-S-V-L-T-P-L-L-L-R-G-L-T-G-S-A-R-R-L-P-V-P-R-A-
K-I-H-S-L;
(SEQ ID NO: 26)
K-K-K-K-K-K-K-K-K-K-K-K-A-A-V-A-L-L-P-A-V-L-L-A-
L-L-A-P-M-S-V-L-T-P-L-L-L-R-G-L-T-G-S-A-R-R-L-P-
V-P-R-A-K-I-H-S-L;
(SEQ ID NO: 27)
K-K-K-K-K-K-K-K-K-K-K-K-K-K-K-K-A-A-V-A-L-L-P-A-
V-L-L-A-L-L-A-P-M-S-V-L-T-P-L-L-L-R-G-L-T-G-S-A-
R-R-L-P-V-P-R-A-K-I-H-S-L;
(SEQ ID NO: 28)
K-K-K-K-A-A-V-A-L-L-P-A-V-L-L-A-L-L-A-P;
(SEQ ID NO: 29)
K-K-K-K-K-K-K-K-A-A-V-A-L-L-P-A-V-L-L-A-L-L-A-P;
(SEQ ID NO: 30)
K-K-K-K-K-K-K-K-K-K-K-K-A-A-V-A-L-L-P-A-V-L-L-A-
L-L-A-P;
(SEQ ID NO: 31)
K-K-K-K-K-K-K-K-K-K-K-K-K-K-K-K-A-A-V-A-L-L-P-A-
V-L-L-A-L-L-A-P;
(SEQ ID NO: 32)
Y-K-K-K-K-K-K-K-K-K-K-K-K-K-K-K-K-A-A-V-A-L-L-P-
A-V-L-L-A-L-L-A-P;
(SEQ ID NO: 33)
K-K-K-K-K-K-K-K-K-K-K-K-K-K-K-K-A-A-V-A-L-L-P-A-
V-L-L-A-L-L-A-P-A-A-V-A-L-L-P-A-V-L-L-A-L-L-A-P;
(SEQ ID NO: 34)
A-A-V-A-L-L-P-A-V-L-L-A-L-L-A-P-K-K-K-K-K-K-K-K-
K-K-K-K-A-A-V-A-L-L-P-A-V-L-L-A-L-L-A-P;
(SEQ ID NO: 35)
K-K-K-K-K-K-K-K-K-K-K-K-K-K-K-K-A-A-V-W-L-L-W-Y-
V-L-L-F-L-L-Y-L;
and
(SEQ ID NO: 36)
K-K-K-K-K-K-K-K-K-K-K-K-K-K-K-K-F-W-V-W-L-L-W-Y-
V-L-L-F-L-L-Y-L.

16. The method of claim 0, wherein the biologically active molecule is selected from the group consisting of: polypeptides; oligonucleotides; plasmids; small molecules; antibodies; antibody fragments; carbohydrates; lipids; glycolipids; antigens; and antigenic peptides.

17. The method of claim 0, wherein the biologically active molecule is selected from the group consisting of: polypeptides; oligonucleotides; and plasmids.

18. The method of claim 0, wherein the oligonucleotides are selected from the group consisting of: coding DNA sequences; antisense DNA sequences; mRNAs, antisense RNA sequences; RNAis; and siRNAs.

19. The method of claim 0, wherein the small molecules are therapeutic agents.

20. The method of claim 0, wherein the molecule is non-covalently bound to the carrier peptide.

21. The method of claim 0, wherein the subject is a human.

22. A method of transporting a biologically active molecule into a mitochondrion of a subject comprising administering to the subject a complex comprising the molecule associated with a carrier peptide; wherein the carrier peptide comprises the sequence:
(SEQ ID NO: 41)
Kₙ-[X₁-X₂-V-X₃-L-L-X₄-X₅-V-L-L-X₆-L-L-X₇-X₈]ₘ
wherein:
X₁-X₈ are independently L, A, W, F, Y, or V;
n is an integer from 0 to 20; and
m is an integer from 1 to 6;
thereby transporting the molecule into the mitochondria.

23. The method of claim 0, wherein the carrier peptide comprises the sequence:
(SEQ ID NO: 38)
[X₁-X₂-V-X₃-L-L-X₄-X₅-V-L-L-X₆-L-L-X₇-X₈]ₚKₙ-[X₁-
X₂-V-X₃-L-L-X₄-X₅-V-L-L-X₆-L-L-X₇-X₈]ₘ
wherein:
X₁-X₈ are independently L, A, W, F, Y, or V;
n is an integer from 0 to 20;
m is an integer from 1 to 6; and
p is an integer from 1 to 6.

24. The method of claim 0 or 0, wherein:
X₁ is A or F;
X₂ is A or W;
X₃ is A or W;
X₄ is P or W;
X₅ is A or Y;
X₆ is A or F;
X₇ is A or Y;
X₈ is P or L.

25. The method of claim 0, wherein the carrier peptide is selected from the group consisting of:
(SEQ ID NO: 24)
K-K-K-K-A-A-V-A-L-L-P-A-V-L-L-A-L-L-A-P-M-S-V-L-
T-P-L-L-L-R-G-L-T-G-S-A-R-R-L-P-V-P-R-A-K-I-H-S-
L;
(SEQ ID NO: 25)
K-K-K-K-K-K-K-K-A-A-V-A-L-L-P-A-V-L-L-A-L-L-A-P-
M-S-V-L-T-P-L-L-L-R-G-L-T-G-S-A-R-R-L-P-V-P-R-A-
K-I-H-S-L;
(SEQ ID NO: 26)
K-K-K-K-K-K-K-K-K-K-K-K-A-A-V-A-L-L-P-A-V-L-L-A-
L-L-A-P-M-S-V-L-T-P-L-L-L-R-G-L-T-G-S-A-R-R-L-P-
V-P-R-A-K-I-H-S-L;
(SEQ ID NO: 27)
K-K-K-K-K-K-K-K-K-K-K-K-K-K-K-K-A-A-V-A-L-L-P-A-
V-L-L-A-L-L-A-P-M-S-V-L-T-P-L-L-L-R-G-L-T-G-S-A-
R-R-L-P-V-P-R-A-K-I-H-S-L;
(SEQ ID NO: 28)
K-K-K-K-A-A-V-A-L-L-P-A-V-L-L-A-L-L-A-P;
(SEQ ID NO: 29)
K-K-K-K-K-K-K-K-A-A-V-A-L-L-P-A-V-L-L-A-L-L-A-P;
(SEQ ID NO: 30)
K-K-K-K-K-K-K-K-K-K-K-K-A-A-V-A-L-L-P-A-V-L-L-A-
L-L-A-P;
(SEQ ID NO: 31)
K-K-K-K-K-K-K-K-K-K-K-K-K-K-K-K-A-A-V-A-L-L-P-A-
V-L-L-A-L-L-A-P;
(SEQ ID NO: 32)
Y-K-K-K-K-K-K-K-K-K-K-K-K-K-K-K-K-A-A-V-A-L-L-P-
A-V-L-L-A-L-L-A-P;
(SEQ ID NO: 33)
K-K-K-K-K-K-K-K-K-K-K-K-K-K-K-K-A-A-V-A-L-L-P-A-
V-L-L-A-L-L-A-P-A-A-V-A-L-L-P-A-V-L-L-A-L-L-A-P;
(SEQ ID NO: 34)
A-A-V-A-L-L-P-A-V-L-L-A-L-L-A-P-K-K-K-K-K-K-K-K-
K-K-K-K-A-A-V-A-L-L-P-A-V-L-L-A-L-L-A-P;
(SEQ ID NO: 35)
K-K-K-K-K-K-K-K-K-K-K-K-K-K-K-K-A-A-V-W-L-L-W-Y-
V-L-L-F-L-L-Y-L;
and
(SEQ ID NO: 36)
K-K-K-K-K-K-K-K-K-K-K-K-K-K-K-K-F-W-V-W-L-L-W-Y-
V-L-L-F-L-L-Y-L.

26. The method of claim 0, wherein the biologically active molecule is selected from the group consisting of: polypeptides; oligonucleotides; plasmids; small molecules; antibodies; antibody fragments; carbohydrates; polysaccharides; lipids; glycolipids; antigens; and antigenic peptides.

27. The method of claim 0, wherein the biologically active molecule is selected from the group consisting of: polypeptides; oligonucleotides; and plasmids.

28. The method of claim 0, wherein the oligonucleotides are selected from the group consisting of: coding DNA sequences; antisense DNA sequences; mRNAs, antisense RNA sequences; RNAis; and siRNAs.

29. The method of claim 0, wherein the small molecules are therapeutic agents.

30. The method of claim 0, wherein the molecule is non-covalently bound to the carrier peptide.

31. The method of claim 0, wherein the subject is a human.

32. A method of transporting a biologically active molecule into a cell comprising contacting the cell with a complex comprising a biologically active molecule associated with a carrier peptide; wherein said carrier peptide comprises the sequence:
(SEQ ID NO: 41)
Kₙ-[X₁-X₂-V-X₃-L-L-X₄-X₅-V-L-L-X₆-L-L-X₇-X₈]ₘ
wherein:
X₁-X₈ are independently L, A, W, F, Y, or V;
n is an integer from 0 to 20; and
m is an integer from 1 to 6;
thereby transporting the molecule into the cell.

33. A method of transporting a biologically active molecule into a mitochondrion comprising contacting the mitochondrion with a complex comprising a biologically active molecule associated with a carrier peptide; wherein said carrier peptide comprises the sequence:
(SEQ ID NO: 41)
Kₙ-[X₁-X₂-V-X₃-L-L-X₄-X₅-V-L-L-X₆-L-L-X₇-X₈]ₘ
wherein:
X₁-X₈ are independently L, A, W, F, Y, or V;
n is an integer from 0 to 20; and
m is an integer from 1 to 6;
thereby transporting the molecule into the mitochondrion.

34. A pharmaceutical composition comprising a carrier peptide comprising the sequence:
(SEQ ID NO: 37)
Kₙ-[X₁-X₂-V-X₃-L-L-X₄-X₅-V-L-L-X₆-L-L-X₇-X₈]ₘ
wherein:
X₁-X₈ are independently L, A, W, F, Y, or V;
n is an integer from 4 to 20; and
m is an integer from 1 to 6.

35. The pharmaceutical composition of claim 0, wherein the composition further comprises a biologically active molecule associated with the carrier peptide.

36. The pharmaceutical composition of claim 0, wherein the biologically active molecule is non-covalently bound to the carrier peptide.

37. A method of modulating expression of a nucleic acid of interest in a subject comprising administering to the subject a complex comprising a carrier peptide and an oligonucleotide, wherein the carrier peptide comprises the sequence:
(SEQ ID NO: 37)
Kₙ-[X₁-X₂-V-X₃-L-L-X₄-X₅-V-L-L-X₆-L-L-X₇-X₈-]ₘ
wherein:
X₁-X₈ are independently L, A, W, F, Y, or V;
n is an integer from 4 to 20; and
m is an integer from 1 to 6.

38. The method of claim 0, wherein the oligonucleotide is selected from the group consisting of: coding DNA sequences; antisense DNA sequences; mRNAs, antisense RNA sequences; RNAis; and siRNAs.

39. The method of claim 0, wherein the oligonucleotide is siRNA.

40. The method of claim 0, wherein the modulating is decreased expression of a nucleic acid of interest.

41. The method of claim 0, wherein the nucleic acid of interest is an endogenous nucleic acid.

42. The method of claim 0, wherein the endogenous nucleic acid is associated with cancer.

43. The method of claim 0, wherein the oligonucleotide is an siRNA targeted to the endogenous nucleic acid.

44. The method of claim 0, wherein the modulating is increased expression of the nucleic acid of interest.

45. A method of delivering a nucleic acid of interest into a cell, the method comprising administering a complex comprising a carrier peptide and a nucleic acid of interest, wherein the carrier peptide comprises the sequence:
(SEQ ID NO: 37)
Kₙ-[X₁-X₂-V-X₃-L-L-X₄-X₅-V-L-L-X₆-L-L-X₇-X₈]ₘ
wherein:
X₁-X₈ are independently L, A, W, F, Y, or V;
n is an integer from 4 to 20; and
m is an integer from 1 to 6.

46. A kit comprising a carrier peptide, wherein the carrier peptide comprising the sequence:
(SEQ ID NO: 37)
Kₙ-[X₁-X₂-V-X₃-L-L-X₄-X₅-V-L-L-X₆-L-L-X₇-X₈]ₘ
wherein:
X₁-X₈ are independently L, A, W, F, Y, or V;
n is an integer from 4 to 20; and
m is an integer from 1 to 6.

47. The kit of claim 0, wherein the kit further comprises a biologically active molecule.

48. The kit of claim 0, wherein the kit further comprises instructions for administering to a subject.

49. The kit of claim 0, wherein the kit further comprises a label that that indicates that the contents are to be administered to a subject with a biologically active molecule.

50. A carrier peptide comprising the sequence:
Kₙ-[(SEQ ID NO: 17)]ₘ
wherein n is an integer from 0 to 20; and
m is an integer from 1 to 6.

51. A carrier peptide comprising the sequence:
[(SEQ ID NO: 17)]ₚ-Kₙ-[(SEQ ID NO: 17)]ₘ
wherein n is an integer from 0 to 20;
m is an integer from 1 to 6; and
p is an integer from 1 to 6.
